(19) United States
(12) Patent Application Publication
(10) Pub. No.: US 2005/0096395 A1
(43) Pub. Date: May 5, 2005

(54) Methods of treating attention deficit/hyperactivity disorder (adhd)

(76) Inventors: Srinivas G Rao, Encinitas, CA (US); Jay D. Kranzler, La Jolla, CA (US)

Correspondence Address: DARBY & DARBY P.C., P. O. BOX 5257, NEW YORK, NY, 10150-5257, US

(21) Appl. No.: 10/504,435

(22) PCT Filed: Feb. 12, 2003
(86) PCT No.: PCT/US03/04095

Related U.S. Application Data
(60) Provisional application No. 60/356,688, filed on Feb. 12, 2002.

(51) Int. Cl. A61K 31/137

(52) U.S. Cl. 514/649

(57) ABSTRACT

The present invention provides a method of treating attention deficit/hyperactivity disorder (AD/HD) and associated tic disorders in an animal subject comprising administering an effective amount of an anti-AD/HD compound or a pharmaceutically acceptable salt thereof. The anti-AD/HD compound useful in the present invention is characterized by ant-AD/HD and anti-tic properties and exhibits at least two distinct pharmacological activities. In particular, the use of milnacipran to treat AD/HD and comorbid tic and psychiatric disorders is disclosed.

### FIELD OF THE INVENTION

The present invention relates to methods for the treatment of attention deficit/hyperactivity disorder (AD/HD). In particular, AD/HD patients, with comorbid tic disorders, are treated with compounds that exhibit both anti-AD/HD and anti-tic properties. The compounds used in the present invention exhibit these two properties in the same molecule and are characterized by at least two distinct pharmacological activities.

### BACKGROUND OF THE INVENTION

Attention deficit/hyperactivity disorder (AD/HD) is among the most common psychiatric disorder in children, with prevalence estimated to be as high as 3-7% in school age children. The disorder is more common in boys than in girls, and often improves over time. However, a significant number of adults are affected as well.

It is widely believed that AD/HD is caused by defects in dopamine transmission, particularly in the mesolimbic and cortical areas that are involved in attention, persistence, and control. Neuroimaging studies also suggest that structural and functional changes in the cortical or limbic regions contribute to the pathophysiology of AD/HD.

Hence, strategies for treating AD/HD are directed primarily at increasing the amount of dopamine within the brain. The most widely used drugs include methylphenidate (Ritalin, Concerta), dextroamphetamine and amphetamine salts (Dexedrine, Adderall, Attendaid), and pemoline (Cylert). All these drugs act by increasing dopamine within the synapse by blocking the dopamine transporter and/or by causing the release of dopamine from the presynaptic terminal.

A second treatment strategy utilizes drugs that act on the noradrenergic system. Two such drugs, clonidine (Catapres) and guanfacine (Tenex) are agonists of the α2 adrenergic receptors, the receptor thought to be involved in the cognitive effects of norepinephrine (NE).

Other agents, such as buproprion, which is thought to increase both dopamine and NE mediated neurotransmission, and venlafaxine (Effexor) which blocks reuptake of serotonin and norepinephrine, have also been used (Adler et al., 1995, Psychopharmacol Bull., 31: 785-8 and Olevera et al., 1996, J Child Adolesc psychopharmacol, 6: 241-50).

The positive effects on attention produced by dopamine stimulating drugs are not observed in all patients. In addition, these drugs produce serious side effects in some patients. For example, these drugs can cause insomnia and appetite reduction, and hence are contraindicated in patients with a history of eating disorders. Patients taking these drugs can also experience a withdrawal or rebound reaction at the end of the day, when blood levels drop. Moreover, since these drugs can be abused, they are not typically used in patients with an history of illicit drug use.

The use of dopamine stimulating drugs is highly controversial in AD/HD patients with concomitant tic disorders or in patients at a risk of developing tic disorders. The increase in dopamine caused by these drugs produces the positive effect on attention and hyperactivity symptoms. However, the increased dopamine is also known to contribute to the pathophysiology of tics. Thus, the increased dopamine can exacerbate an existing tic disorder or cause the onset of tics in patients who previously did not exhibit any symptoms of tic disorders.

Although the drugs that act on the noradrenergic system have a better side effect profile compared to dopamine stimulating drugs and are effective against tic disorders, this class of drugs is not particularly effective in improving attention and/or hyperactivity symptoms in AD/HD patients.

Another drawback of the current treatments for AD/HD is that they are not particularly effective in treating psychiatric disorders. Very often, AD/HD patients are diagnosed with comorbid psychiatric disorders. In these patients, the current therapy is often supplemented with anti-depressants to treat the comorbid psychiatric disorders. The addition of another therapeutic agent into the patient's treatment regimen increases the risk of development of side effects and decreases patient compliance.

Due to the reasons presented above, there is a demand for more effective agents to treat AD/HD patients, and in particular those who suffer from associated psychiatric and tic disorders. The ideal agent would treat the underlying disorder and/or reduce the symptoms associated with AD/HD and comorbid psychiatric and tic disorders, act satisfactorily whether given orally or parenterally, and produce minimal or no side effects.

### SUMMARY OF THE INVENTION

In one aspect, the invention provides a method of treating attention deficit/hyperactivity disorder (AD/HD) and optionally tic disorders associated with AD/HD in an animal subject including a human. The method generally involves administering to an animal subject suffering from AD/HD and comorbid tic disorders an effective amount of an anti-AD/HD compound or a pharmaceutically acceptable salt thereof. The anti-AD/HD compounds that are useful in the present invention are characterized by anti-AD/HD and anti-tic properties and exhibit at least two distinct pharmacological activities.

The invention also provides a method of treating attention deficit/hyperactivity disorder (AD/HD) and optionally tic disorders associated with AD/HD involving the administration to an animal subject suffering from AD/HD and optionally comorbid tic disorders an effective amount of an anti-AD/HD (≠DA, NE) compound or a pharmaceutically acceptable salt thereof. The anti-AD/HD (≠DA, NE) compounds that are useful in the present invention are characterized by anti-AD/HD and anti-tic properties, at least two distinct pharmacological activities, and the lack of both dopamine and norepinephrine stimulating activities in the same molecule.

Another aspect of the invention provides a method of treating AD/HD and optionally tic disorders associated with AD/HD involving the administration to an animal subject suffering from AD/HD and optionally comorbid tic disorders an effective amount of milnacipran or a pharmaceutically acceptable salt thereof.

In yet another aspect, the invention provides a kit comprising a compound useful in the present invention packaged in association with instructions teaching a method of using the compound according to one or more of the above-described methods. The kit can contain the compound packaged in unit dosage form. In one embodiment, milnacipran or a pharmaceutically acceptable salt thereof is included in the kit.

### DETAILED DESCRIPTION OF THE PREFERRED EMBODIMENTS

Abbreviations

AD/HD attention deficit/hyperactivity disorder

AMPA alpha-amino-3-hydroxy-5-methylisoxazole-4-proprionic acid

GABA γ-amino butyric acid

5-HT serotonin

NE norepinephrine

NMDA N-methyl D-aspartate

SNRIs dual serotonin norepinephrine reuptake inhibitors

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano. When a substituent is keto (i.e., ═O) or thioxo (i.e., ═S) group, then 2 hydrogens on the atom are replaced.

“Therapeutically effective amount” is intended to include an amount of a compound useful in the present invention or an amount of the combination of compounds claimed, e.g., to treat or prevent cognitive dysfunctions or treat the symptoms of cognitive dysfunctions in a host. The combination of compounds is preferably a synergistic combination. Synergy, as described for example by Chou and Talalay, Adv. Enzyme Regul. 22: 27-55 (1984), occurs when the effect (in this case, treatment or prevention of cognitive dysfunctions) of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

The term “alkyl” refers to a monoradical branched or unbranched saturated hydrocarbon chain preferably having from 1 to 40 carbon atoms, more preferably 1 to 10 carbon atoms, and even more preferably 1 to 6 carbon atoms. This term is exemplified by groups such as methyl, ethyl, n-propyl, iso-propyl, n-butyl, iso-butyl, sec-butyl, n-hexyl, n-decyl, tetradecyl, and the like.

The alkyl can optionally be substituted with one or more alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “alkylene” refers to a diradical branched or unbranched saturated hydrocarbon chain preferably having from 1 to 40 carbon atoms, more preferably 1 to 10 carbon atoms, and even more preferably 1 to 6 carbon atoms. This term is exemplified by groups such as methylene, ethylene, n-propylene, iso-propylene, n-butylene, iso-butylene, sec-butylene, n-hexylene, n-decylene, tetradecylene, and the like.

The alkylene can optionally be substituted with one or more alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “alkoxy” refers to the groups alkyl-O-, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The alkyoxy can optionally be substituted with one or more alkyl, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like.

The aryl can optionally be substituted with one or more alkyl, alkoxy, halo, haloalkyl, hydroxy, hydroxyalkyl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkoxy, halo, haloalkyl, hydroxy, hydroxyalkyl, aryl, heteroaryl, heterocycle, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkoxy, halo, haloalkyl, hydroxy, hydroxyalkyl, aryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “heterocycle” refers to a saturated or partially unsaturated ring system, containing at least one heteroatom selected from the group oxygen, nitrogen, and sulfur, and optionally substituted with alkyl or C(═O)OR^{b}, wherein R^{b} is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkoxy, halo, haloalkyl, hydroxy, hydroxyalkyl, aryl, heteroaryl, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

Another class of heterocyclics is known as “crown compounds” which refers to a specific class of heterocyclic compounds having one or more repeating units of the formula [—(CH₂—)ₐA-] where a is equal to or greater than 2, and A at each separate occurrence can be O, N, S or P. Examples of crown compounds include, by way of example only, [—(CH₂)₃—NH—]₃, [—((CH₂)₂—O)₄—((CH₂)₂—NH)₂] and the like. Typically such crown compounds can have from 4 to 10 heteroatoms and 8 to 40 carbon atoms.

The term “alkanoyl” refers to C(═O)R, wherein R is an alkyl group as previously defined.

The term “alkoxycarbonyl”0 refers to C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “nitro” refers to —NO₂; the term “trifluoromethyl” refers to —CF₃; the term “trifluoromethoxy” refers to —OCF₃; the term “cyano” refers to —CN; and the term “hydroxy” refers to —OH.

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

“Prodrugs” are intended to include any covalently bonded substances, which release the active parent drug or other formulas or compounds of the present invention in vivo when such prodrug is administered to a mammalian subject. Prodrugs of a compound of the present invention, for example milnacipran, are prepared by modifying functional groups present in the compound in such a way that the modifications are cleaved, either in routine manipulation in vivo, to the parent compound. Prodrugs include compounds of the present invention wherein the hydroxy or amino group is bonded to any group that, when the prodrug is administered to a mammalian subject, cleaves to form a free hydroxyl or free amino, respectively. Examples of prodrugs include, but are not limited to, acetate, formate and benzoate derivatives of alcohol and amine functional groups in the compounds of the present invention, and the like.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic. A specific metabolic pathway includes the glucuronide conjugation.

The term “dual serotonin norepinephrine reuptake inhibitor compound” or SNRI refers to the well-recognized class of anti-depressant compounds that selectively inhibit reuptake of both serotonin and norepinephrine. Common SNRI compounds include, but are not limited to, venlafaxine, duloxetine, and milnacipran.

The terms “NE≧5-HT SNRI” and “NE>5-HT SNRI” refer to particular subclasses of SNRI compounds that are useful in the methods and kits of the present invention, as will be described in more detail herein.

As mentioned above, the NE≧5-HT SNRI compounds useful in the methods and kits of the invention include compounds that inhibit norepinephrine reuptake to a greater extent than serotonin reuptake, as well as compounds that inhibit the reuptake of these two monoamines to an equivalent extent. In one embodiment of the invention, the NE≧5-HT SNRI compounds have a ratio of inhibition of norepinephrine reuptake to serotonin reuptake (“NE:5-HT”) in the range of about 1-100:1. In a particular embodiment, the compounds are NE>5-HT SNRI compounds, i.e., compounds that inhibit norepinephrine reuptake to a greater extent than serotonin reuptake. Such NE>5-HT SNRI compounds generally have a NE:5-HT in the range of about 1.1-100:1. That is, such NE>5-HT SNRI compounds are at least about 1.1 to about 100 times more effective at inhibiting norepinephrine reuptake than serotonin reuptake. NE>5-HT SNRI compounds having a NE:5-HT ratio in the range of about 2:1 to about 10:1 may be particularly effective.

Various techniques are known in the art to determine the NE:5-HT of a particular SNRI. In one embodiment, the ratio can be calculated from IC₅₀ data for NE and 5-HT reuptake inhibition. For example, it has been reported that for milnacipran the IC₅₀ of norepinephrine reuptake is 100 nM, whereas the IC₅₀ serotonin reuptake inhibition is 200 nM. See Moret et al., 1985, Neuropharmacology 24(12): 1211-1219; Palmier et al., 1989, Eur J Clin Pharmacol 37: 235-238. Therefore, the NE:5-HT reuptake inhibition ratio for milnacipran based on this data is 2:1. Of course, other IC values such as IC₂₅, IC₇₅, etc. could be used, so long as the same IC value is being compared for both norepinephrine and serotonin. The concentrations necessary to achieve the desired degree of inhibition (i.e., IC value) can be calculated using known techniques either in vivo or in vitro. See Sanchez et al., 1999, Cellular and Molecular Neurobiology 19(4): 467-489; Turcotte et al., 2001, Neuropsychopharmacology 24(5): 511-521; Moret et al., 1985, Neuropharmacology 24(12): 1211-1219; Moret et al., 1997, J. Neurochem. 69(2): 815-822; Bel et al., 1999, Neuropsychopharmacology 21(6): 745-754; and Palmier et al., 1989, Eur J Clin Pharmacol 37: 235-238.

The NE:5-HT of a particular SNRI also can be calculated using equilibrium dissociation constants (K_{D}'S) for norepinephrine and serotonin transporters as described in Tatsumi et al., 1997, European Journal of Pharmacology 340: 249-258. For example, a NE>5-HT SNRI compound with a K_{D} of 2 nM for the norepinephrine transporter and a K_{D} of 8 nM for the serotonin transporter has an NE:5-HT of 4:1.

Yet another means for determining the NE:5-HT of a particular SNRI involves measuring the affinity (Kᵢ) of the SNRI for the norepinephrine and serotonin transporters as described in Owens et al., 1997, JPET 283: 1305-1322. For example, a NE>5-HT SNRI compound with a Kᵢ of 1 nM for the norepinephrine transporter and a Kᵢ of 20 nM for the serotonin transporter has an NE:5-HT of 20:1.

A specific example of a NE≧5-HT SNRI compound that can be used to practice the present invention is milnacipran. Additional NE≧5-HT SNRI compounds that can be used to practice the present invention include, by way of example and not limitation, any of the aminocyclopropane derivatives disclosed in the following references that inhibit norepinephrine reuptake to an equivalent or greater extent than serotonin reuptake (i.e., that have a NE:5-HT ratio that is 1:1): W095/22521; U.S. Pat. No. 5,621,142; Shuto et al., 1995, J. Med. Chem. 38: 2964-2968; Shuto et al., 1996, J. Med. Chem. 39: 4844-4852; Shuto et al., 1998, J. Med. Chem. 41: 3507-3514; Shuto et al., 2001, Jpn. J. Pharmacol. 85: 207-213; Noguchi et al., 1999, Synapse 31: 87-96; and U.S. Pat. No. 4,478,836. All of these references are hereby incorporated herein by reference in their entireties.

In a specific embodiment of the invention, the NE>5-HT compound is milnacipran. The chemical structure of milnacipran, cis-(±)-2-(aminomethyl)-N,N-diethyl-1-phenyl-yclopropanecarboxamide, is as follows: 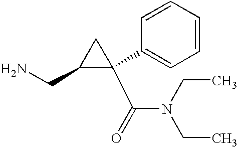
Milnacipran is also known in the art as F2207, TN-912, dalcipran, midalcipran, and midalipran. The NE:5-HT ratio of milnacipran is about 2:1. See Moret et al., 1985, Neuropharmacology 24(12): 1211-1219; Palmier et al., 1989, Eur J Clin Pharmacol 37: 235-238. Milnacipran and methods for its synthesis are described in U.S. Pat. No. 4,478,836, which is hereby incorporated by reference in its entirety. Additional information regarding milnacipran may be found in the Merck Index, 12^{th} Edition, at entry 6281. Quite significantly, milnacipran has been used as an antidepressant in approximately 400,000 patients, and is known to be non-toxic in humans. In clinical trials at dosages of 100 mg/day or 200 mg/day, milnacipran was well tolerated and usually produced no more adverse effects than placebo (Spencer and Wilde, 1998, Drugs 56(3): 405-427).

Those of skill in the art will recognize that NE≧5-HT SNRI compounds such as milnacipran may exhibit the phenomena of tautomerism, conformational isomerism, geometric isomerism and/or optical isomerism. It should be understood that the invention encompasses any tautomeric, conformational isomeric, optical isomeric and/or geometric isomeric forms of the NE≧5-HT SNRI compounds having one or more of the utilities described herein, as well as mixtures of these various different forms. For example, as is clear from the above structural diagram, milnacipran is optically active. It has been reported in the literature that the dextrogyral enantiomer of milnacipran is about twice as active in inhibiting norepinephrine and serotonin reuptake than the racemic mixture, and that the levrogyral enantiomer is much less potent (see, e.g., Spencer and Wilde, 1998, supra; Viazzo et al., 1996, Tetrahedron Lett. 37(26): 4519-4522; Deprez et al., 1998, Eur. J. Drug Metab. Pharmacokinet. 23(2) : 166-171). Accordingly, milnacipran may be administered in entantiomerically pure form (e.g., the pure dextrogyral enantiomer) or as a mixture of dextogyral and levrogyral enantiomers, such as a racemic mixture. Unless specifically noted otherwise, the term “milancipran” as used herein refers to both enantiomerically pure forms of milnacipran as well as to mixtures of milnacipran enantiomers. Methods for separating and isolating the dextro- and levrogyral enantiomers of milnacipran and other NE≧5-HT SNRI compounds are well-known (see, e.g., Grard et al., 2000, Electrophoresis 2000 21: 3028-3034).

It will also be appreciated that in many instances the NE≧5-HT SNRI compounds may metabolize to produce active NE≧5-HT SNRI compounds. The use of active metabolites is also within the scope of the present invention.

It has been reported that milnacipran and its derivatives have antagonistic properties at the NMDA receptor. See Shuto et al., 1995, J. Med. Chem. 38: 2964-2968; Shuto et al., 1996, J. Med. Chem. 39: 4844-4852; Shuto et al., 1998, J. Med. Chem. 41: 3507-3514; and Shuto et al., 2001, Jpn. J Pharmacol. 85: 207-213. As a consequence, one particularly useful embodiment of the invention includes NE v 5-HT SNRI compounds that also have NMDA antagonistic properties. The NE≧5-HT SNRI compounds with NMDA receptor antagonistic properties can have IC₅₀ values from about 1 nM-100 μM. For example, milnacipran has been reported to have an IC₅₀ value of about 6.3 μM. The NMDA receptor antagonistic properties of milnacipran and its derivatives are described in Shuto et al., 1995, J. Med. Chem., 38: 2964-2968; Shuto et al., 1996, J. Med. Chem. 39: 4844-4852; Shuto et al., 1998, J. Med. Chem. 41: 3507-3514; and Shuto et al., 2001, Jpn. J Pharmacol. 85: 207-213. Methods for determining the antagonism and affinity for antagonism are disclosed in Shuto et al., 1995, J. Med. Chem. 38: 2964-2968; Shuto et al., 1996, J. Med. Chem. 39: 4844-4852; Shuto et al., 1998, J. Med. Chem. 41: 3507-3514; Noguchi et al., 1999, Synapse 31: 87-96; and Shuto et al., 2001, Jpn. J. Pharmacol. 85: 207-213. Aminocyclopropane derivatives disclosed in WO95/22521; U.S. Pat. No. 5,621,142; Shuto et al., 1995, J. Med. Chem. 38: 2964-2968; Shuto et al., 1996, J. Med. Chem. 39: 4844-4852; Shuto et al., 1998, J. Med. Chem. 41: 3507-3514; Noguchi et al., 1999, Synapse 31: 87-96; and Shuto et al., 2001, Jpn. J. Pharmacol. 85: 207-213 that inhibit NE reuptake equal to or greater than 5-HT reuptake and have NMDA antagonistic properties can be used to practice the present invention. These references are hereby incorporated by reference in their entirety.

Quite surprisingly, the present inventors have discovered that the NE≧5-HT SNRI subclass of SNRI compounds are effective in treating AD, HD, tics, or a combination thereof, when administered alone (or in combination with other compounds that are not neurotransmitter precursors, as will be discussed in more detail, below). Thus, in one embodiment of the invention, the NE≧5-HT SNRI compound is administered alone, or in combination with a compound other than a neurotransmitter precursor such as phenylalanine, tyrosine and/or tryptophan.

The NE≧5-HT SNRI compounds, such as, for example, milnacipran, can be administered adjunctively with other active compounds. By adjunctive administration is meant simultaneous administration of the compounds, in the same dosage form, simultaneous administration in separate dosage forms, and separate administration of the compounds.

The NE≧5-HT SNRI compounds can be administered therapeutically to achieve a therapeutic benefit or prophylactically to achieve a prophylactic benefit. By therapeutic benefit is meant eradication or amelioration of the underlying disorder being treated, e.g., eradication or amelioration of the underlying disorder, and/or eradication or amelioration of one or more of the physiological symptoms associated with the underlying disorder such that the patient reports an improvement in feeling or condition, notwithstanding that the patient may still be afflicted with the underlying disorder.

For therapeutic administration, the NE≧5-HT SNRI compound typically will be administered to a patient already diagnosed with the particular indication being treated.

For prophylactic administration, the NE≧5-HT SNRI compound may be administered to a patient at risk of developing AD, HD, tics, or a combination thereof or to a patient reporting one or more of the physiological symptoms of AD, HD, tics, or a combination thereof, even though a diagnosis of AD, HD, tics, or a combination thereof may not have yet been made. Alternatively, prophylactic administration may be applied to avoid the onset of the physiological symptoms of the underlying disorder, particularly if the symptom manifests cyclically. In this latter embodiment, the therapy is prophylactic with respect to the associated physiological symptoms instead of the underlying indication. For example, the NE≧5-HT SNRI compound could be prophylactically administered prior to bedtime to avoid the sleep disturbances associated with AD, HD, tics, or a combination theteof.

### AD/HD, Tic Disorders, and Psychiatric Disorders

The present invention provides methods and kits for treating animal subjects, in particular humans, suffering from AD/HD. The DSM-IV-TR™ defines AD/HD as a persistent pattern of inattention and/or hyperactivity-impulsivity that is more frequently displayed and more severe than is typically observed in individuals at a comparable level of development. In AD/HD patients, some impairment from the symptoms are observed in at least two settings, for example at home and at school or work. One of the diagnostic criteria for AD/HD is the presence of six or more of the following symptoms of inattention for a period of 6 months such that it is maladaptive and inconsistent with developmental level: (i) failure to give close attention to details or making careless mistakes in school work, work, or other activities; (ii) difficulty sustaining attention in tasks or play activities; (iii) does not seem to listen when spoken to directly; (iv) does not follow through on instructions and fails to finish school work, chores, or duties in workplace; (v) difficulty organizing tasks and activities; (vi) avoids, dislikes, or is reluctant to engage in tasks that require sustained mental effort; (vii) loses things necessary for tasks or activities; (viii) easily distracted by extraneous stimuli; and (ix) forgetful in daily activities. Another diagnostic criteria is the evaluation of the following symptoms of hyperactivity-impulsivity for a period of 6 months such that it is maladaptive and inconsistent with developmental level: (i) fidgets with hands or feet or squirms in seat; (ii) leaves seat in classroom or in other situation in which remaining seated is expected; (iii) runs about or climbs excessively in situations in which it is inappropriate; (iv) difficulty playing or engaging in leisure activities quietly; (v) often “on the go” or acts as if “driven by a motor;” (vi) talks excessively; (vii) blurts out answers before questions have been completed; (viii) has difficulty awaiting turn; and (ix) interrupts or intrudes on others. There are three subtypes of AD/HD: AD/HD, combined type; AD/HD, predominantly inattentive type; and AD/HD, predominantly hyperactive-impulsive type. The AD/HD, combined type diagnosis is used if six or more symptoms for both inattention and hyperactivity-impulsivity have persisted for at least six months. A diagnosis of AD/HD, predominantly inattentive type is made of six or more symptoms for inattention (but fewer than six symptoms of hyperactivity-impulsivity) have persisted for at least six months. The AD/HD, predominantly hyperactive-impulsive type diagnosis is used when six or more symptoms for hyperactivity-impulsivity (but fewer than six symptoms of inattention) have persisted for at least six months. The methods and kits of the present invention are useful in treating all three subtypes of AD/HD.

In particular, the compounds of the present invention are useful in treating a subpopulation of AD/HD patients suffering from comorbid tic disorders. Comorbid tic disorders, including Tourette's syndrome, are diagnosed in a subpopulation of AD/HD patients. A tic is a sudden, rapid, recurrent, nonrhythmic, stereotyped motor movement or vocalization. Motor and vocal tics may be simple (involving only a few muscles or simple sounds) or complex (involving multiple groups of muscles recruited in orchestrated bouts or words or sentences). AD/HD patients may be diagnosed with comorbid Tourette's syndrome, chronic motor tic disorder, chronic vocal tic disorder, or transient tics disorder. Tourettes' syndrome is characterized by both multiple motor and one or more vocal tics present during the illness, although not necessarily concurrently. Chronic motor or vocal tic disorders are characterized by single or multiple motor or vocal tics, but not both, present during the illness. In Tourette's syndrome, chronic motor tic disorder, and chronic vocal tic disorder the tics occur many times a day (usually in bouts) nearly every day or intermittently throughout a period of more than 1 year, and during this period there is no tic-free period of more than three consecutive months. In transient tic disorder the single or multiple motor tics and/or vocal tics occur many times a day, nearly every day for at least four weeks, but for no longer than twelve consecutive months.

Some AD/HD patients are diagnosed with concomitant tic disorders along with AD/HD. Whereas, in some AD/HD patients the tic disorders are a direct physiological consequence of the central nervous system stimulants used in the treatment of AD/HD. The central nervous stimulants that can have this consequence include methylphenidate, pemoline, and dextroamphetamine. The central nervous stimulants can either cause tic disorders in AD/HD patients or exacerbate an existing concomitant tic disorder. The term “comorbid tic disorder” as used herein means both a concomitant tic disorder diagnosed in an AD/HD patient and a tic disorder in an AD/HD patient induced by the current AD/HD therapy. In one embodiment of the invention, a compound useful in the present invention is administered to a patient diagnosed with both AD/HD and a concomitant tic disorder. In another embodiment, the compound is administered to a patient diagnosed with AD/HD who has developed a tic disorder due to the current AD/HD therapy. A significant advantage of the methods of the present invention is not only the ability to treat comorbid tic disorders, but also to treat the AD/HD without exacerbating or inducing a tic disorder.

Further, the compounds of the present invention are useful in treating the subpopulation of AD/HD patients suffering from both comorbid tic and psychiatric disorders. Psychiatric disorders associated with AD/HD include oppositional-defiant disorder, conduct disorder depressive disorder, anxiety disorder, obsessive-compulsive disorder, and learning disorders. Oppositional-defiant disorder is a recurrent pattern of negativistic, defiant, disobedient, and hostile behaviors toward authority figures that persists for at least six months. These behaviors occur more frequently than is typically observed in individuals of comparable age and developmental level and leads to significant impairment in social, academic, or occupational functioning. Conduct disorder is a repetitive and persistent pattern of behavior in which the basic rights of others or major age-appropriate societal norms or rules are violated. Depressive disorders are characterized by major depressive episodes without a history of manic, mixed, or hypomaniac episodes. Anxiety disorder is characterized by excessive worry, i.e., excessive concerns about real life concerns. The features of obsessive-compulsive disorder include recurrent obsessions or compulsions that are severe enough to be time consuming or cause marked distress or significant impairment. Obsessions are persistent ideas, thoughts, impulses or images that are experienced as intrusive and inappropriate and that cause marked anxiety or distress. Compulsions are repetitive behaviors or mental acts the goal of which is to prevent or reduce anxiety or distress, not to provide pleasure or gratification. Learning disorders are diagnosed when the individual's achievement on individually administered, standardized tests in reading, mathematics, or written expression is substantially below that expected for age, schooling, and level of intelligence. The learning problems significantly interfere with academic achievement or activities of daily living that require reading, mathematical, or writing skills. One or more psychiatric disorders described above may be comorbid in AD/HD patients. There are various means to diagnose these psychiatric disorders. These means include various psychological and behavioral evaluations. Such means are well described in the scientific literature, for example in Diagnostic and Statistical Manual of Mental Disorders, Fourth Edition.

The art provides various means for diagnosing 20 AD/HD and comorbid tic and/or psychiatric disorders.

Described above are some means for diagnosing these disorders. The diagnostic criteria described above were obtained from Diagnostic and Statistical Manual of Mental Disorders, Fourth Edition. It would be apparent to one of skill in the art that, in addition to the diagnostic criteria described above, different diagnostic criteria described in other scientific literature may also be used.

### Pharmacological Activities

The compounds useful in the present invention can exhibit anti-AD/HD, anti-tic, and anti-psychiatric properties. These compounds demonstrate these properties by two or more pharmacological activities.

While not intending to be bound by any particular theory of operation, it is believed that the pharmacological activities that are related to the anti-AD/HD properties include dopamine stimulation activity and increased norepinephrine activity in the central nervous system. Some of the pharmacological activities related to the anti-tic properties include dopamine receptor antagonistic activity, increase in GABA activity in the central nervous system, decrease in glutaminergic activity, or α2 agonistic activity. An increase in serotonin activity in the central nervous system is believed to be one of the pharmacological activities related to the anti-psychiatric properties of the compounds of the present invention.

The dopamine stimulation activity includes, but is not limited to, blocking the dopamine transporter (DAT) such that dopamine reuptake is inhibited or causing the release of dopamine from the presynaptic terminal. The ability of a compound to block the DAT or increase release of dopamine can be determined using several techniques known in the art. For example Gainetdinov et al., 1999, Science, 283: 397-401, describes a technique in which the extracellular dopamine concentration in the striatum can be measured using microdialysis. To determine the ability of a compound to block the DAT or increase the release of dopamine, the extracellular concentration of dopamine can be measured before and after administration of said compound. A statistically significant increase in dopamine levels post-administration of the compound being tested indicates that said compound inhibits the reuptake of dopamine or increases the release of dopamine. The ability to block the DAT can also be quantified with inhibitory concentration (IC) values, like IC₅₀, at the dopamine transporter. Several techniques for determining IC values are described in the art. For example, see Rothman et al., 2000, Synapse, 35: 222-227. The compounds useful in the present invention can have IC₅₀ values in the range of 0.1 nM to 600 μM. In particular, the compounds have IC₅₀ values of 0.1 nM to 100 μM.

The norepinephrine stimulation activity in the central nervous system can be related to, but not limited to, either inhibition of norepinephrine reuptake or α2 agonistic activity. The inhibition of norepinephrine reuptake can be via the blocking of the norepinephrine transporter (NET). The blocking of the NET by a particular compound can be studied using cell lines transfected with NET. For example, see Galli et al., 1995, The Journal of Experimental Biology, 198: 2197-2212. In one embodiment, K₁ values at the NET are used to determine the inhibition of norepinephrine reuptake by specific compounds. The compounds useful in the present invention can have K₁ values in the range of 1.5 nmol/l to 10 μmol/l. Compounds with K₁ values in the range of 100 nmol/l to 700 nmol/l are particularly useful.

The term “α2 agonistic activity” refers to partial or complete activation of the α2 receptor via binding to the α2 receptor. This activity can also include partial or complete activation of any biological response associated with the binding of norepinephrine to the α2 receptor. “α2 receptor” refers to a family of extracellular receptors which specifically bind norepinephrine, epinephrine, and their analogs. See Docherty, 1998, European Journal of Pharmacology, 361: 1-15. The term also refers to isoforms of α2 receptor, recombinant α2 receptor, and mutated α2 receptor. Several techniques are known in the art to determine the α2 agonistic activities of compounds. The α2 agonistic properties of particular compounds can be ascertained by determining EC₅₀ (concentration causing 50% of the maximal effect) values as described in Jansson et al., 1999, European Journal of Pharmacology, 374: 137-146. Suitable compounds can have an EC₅₀ value in the range of 1 nM to 5000 nM, the EC₅₀ value being determined using the technique described in Jansson et al., 1999. In particular, compounds with EC₅₀ values in the range of 5 nM to 3500 nM are useful, the EC₅₀ value being determined using the technique described in Jansson et al., 1999. In the present invention, compounds with either full or partial agonistic activity at the α2 receptor are useful.

The term “dopamine antagonistic activity” refers to partial or complete inhibition (antagonism) of the dopamine receptor agonist such as dopamine to a dopamine receptor. This term also refers to partial or complete inhibition of any biological response associated with the binding of a dopamine receptor to an agonist. “Dopamine receptor” refers to a family of extracellular receptors which specifically bind dopamine, and their analogs (Vallone et al., 2000, Neuroscience and Biobehavioral Reviews, 24: 125-132). The dopamine receptors can also bind norepinephrine and epinephrine at high concentrations. For example, see Newman-Tancredi et al., 1997, European Journal of Pharmacology, 319: 379-383. The term also refers to isoforms of dopamine receptor, recombinant dopamine receptor, and mutated dopamine receptor. Several techniques are known in the art to determine the dopamine antagonistic activity of specific compounds. For example see Fici et al., 1997, Life Sciences, 60: 1597-1603 and Lau et al., 1997, Gen. Pharmac., 29: 729-736. Compounds with IC₅₀ values in the range of 0.1 nM to 100 μM, in particular 0.2 nM to 10 μM, are useful.

One means for achieving an increase in GABA activity in the central nervous system is through the use of GABA agonists. The term “GABA agonist” refers to any composition or compound which partially or completely activates the GABA receptor via binding to the GABA receptor. This term also refers to any composition or compound which partially or completely activates the biological response associated with the binding of GABA to the GABA receptor. “GABA receptor” refers to a family of extracellular receptors which specifically bind GABA and their analogs. Chebib et al., 1999, Clinial and Experimental Pharmacology and Physiology, 26: 937-940. The term also refers to isoforms of GABA receptor, recombinant GABA receptor and mutated GABA receptor. Several techniques are known in the art to determine the GABA agonistic activities of compounds. For example, see Hill-Venning et al., 1996, Neuropharmacology, 35: 1209-1222. In one embodiment, EC₅₀ values at the GABA receptor can be calculated in the presence of GABA, as described in Hill-Venning et al., 1996, to determine the GABA agonistic activity of specific compounds. Suitable compounds have EC₅₀ values in the range of 50 nM to 100 μM, these values being determined in the manner described in Hill-Venning et al., 1996. In the present invention, compounds with either full or partial agonistic activity at the GABA receptor are useful.

The decrease in glutaminergic activity can be achieved through the use of NMDA receptor antagonists or AMPA/kainate antagonists. “N-methyl D-aspartate (NMDA) receptor antagonist” refers to any composition or compound which partially or completely inhibits (antagonizes) the binding of a NMDA receptor agonist such as glutamate or NMDA to a NMDA receptor. A “NMDA receptor antagonist” also refers to any composition or compound which inhibits any biological response associated with the binding of a NMDA receptor to an agonist. “NMDA receptor” refers to a family of extracellular receptors which specifically bind glutamate, NMDA, and their analogs. See Cull-Candy et al., 2001, Current Opinions in Neurobiology, 11: 327-335 and Nankai et al., 1996, Neurochem Int, 29: 529-542. The term also refers to isoforms of NMDA receptor, recombinant NMDA receptor, and mutated NMDA receptor. Several techniques are known in the art to determine the antagonistic properties at the NMDA receptor. For example, see Shuto et al., 1995, J. Med. Chem., 38: 2964-2968; Shuto et al., 1996, J. Med. Chem., 39: 4844-4852; Shuto et al., 1998, J. Med. Chem., 41: 3507-3514; and Shuto et al., 2001, Jpn. J. Pharmacol., 85: 207-213. IC values (for example IC₂₅, IC₅₀, IC₇₅, etc) or K₁ values can be used to quantify the NMDA antagonistic properties of compounds. Compounds with IC₅₀ values at the NMDA receptor of about 1 nM-100 μM are useful. In one aspect of the invention, it is preferred that the compound employed exhibit reversible, low affinity (K₁>0.7 micromolar) binding for the NMDA receptor.

“AMPA/kainate receptor antagonist” refers to any composition or compound which partially or completely inhibits (antagonizes) the binding of an AMPA/kainate receptor agonist such as glutamate, AMPA, or kainic acid to an AMPA/kainate receptor. An “AMPA/kainate receptor antagonist” also refers to any composition or compound which inhibits any biological response associated with the binding of an AMPA/kainate receptor to an agonist. “AMPA/kainate receptor” refers to a family of extracellular receptors which specifically bind glutamate, AMPA, kainic acid, and their analogs. Franciosi, 2001, CMLS, Cell. Mol. Life Sci., 58: 921-930. The term also refers to isoforms of AMPA/kainate receptor, recombinant AMPA/kainate receptor, and mutated AMPA/kainate receptor. Several techniques are known in the art to determine the antagonistic properties at the AMPA/kainate receptor. For example, see Bleakman et al., 1996, Neuropharmacology, 35: 1689-1702. IC values (for example IC₂₅, IC₅₀, IC₇₅, etc) or K₁ values can be used to quantify the AMPA/kainate antagonistic properties of compounds. Compounds with IC₅₀ values at the AMPA/kainate receptor of about 0.1 nM to 500 nM are useful in the present invention.

One means for achieving increased serotonin activity is via inhibition of serotonin reuptake. The ability of a compound to inhibit reuptake of serotonin can be measured using techniques known in the art. For example, see Sanchez et al., 1999, Cellular and Molecular Neurobiology 19(4): 467-489; Turcotte et al., 2001, Neuropsychopharmacology, 24(5): 511-521; Moret et al., 1985, Neuropharmacology 24(12): 1211-1219; Moret et al., 1997, J. Neurochem., 69(2): 815-822; Bel et al., 1999, Neuropsychopharmacology, 21(6): 745-754; and Palmier et al., 1989, Eur J Clin Pharmacol, 37: 235-238. In one aspect of the invention, IC values are used to quantify the ability of a compound to inhibit the reuptake of serotonin. In the present invention compounds with IC₅₀ values in the range of 0.1 nM to 500 nM are particularly useful.

The anti-AD/HD, anti-tic, and anti-psychiatric properties have been described herein as being related to specific pharmacological activities. It will be apparent to one of skill in the art that these properties can be related to other pharmacological activities not described in the present application.

The compounds of the present invention treat AD/HD, tic disorders, and psychiatric disorders by acting on multiple neurotransmitters. One of the advantages of the present invention is the presence of multiple pharmacological activities in one compound. Thus, one agent can be administered to treat both AD/HD and the comorbid disorders. Previously, for example, an AD/HD patient suffering from comorbid tic and psychiatric disorders would have been administered a dopamine stimulating drug for the treatment of AD/HD, a norepinephrine stimulating drug for tics, and an anti-depressant for the psychiatric disorder. Patient compliance was often low as the patient had to self-administer three different drugs each day. Also, the patient was at a risk of developing side effects caused by each of the three drugs. In the present invention, these problems are avoided by the administration of one compound with multiple pharmacological activities. Patient compliance improves as the patient now has to be administered fewer medications and the side effect profile of the treatment improves as the number of medications administered to the AD/HD patient is reduced.

### Treatment of AD/HD, Tic Disorders, and Psychiatric Disorders

In the present invention, a therapeutically effective amount of an anti-AD/HD compound is used to treat the subpopulation of AD/HD patients suffering from comorbid tic disorders. The term “anti-AD/HD compound” as used herein refers to a class of compounds with anti-AD/HD and anti-tic properties. This class of compounds exhibits these two properties by at least two distinct pharmacological activities. Thus, the anti-AD/HD compounds of the present invention do not include compounds like clonidine that exhibit both anti-AD/HD and anti-tic properties, but produces these effects by only one pharmacological activity, i.e. α2 agonistic activity.

In one embodiment of the invention, the compounds used to practice the invention are a subclass of anti-AD/HD compounds that do not exhibit both dopamine and norepinephrine stimulation activity in the same compound. That is, if a particular compound in this subclass exhibits increased dopamine activity, then this compound will not exhibit increased norepinephrine activity, and vice versa. This subclass of compounds is referred to herein as “anti-AD/HD (≠DA, NE) compounds.” This subclass of compounds is used to treat AD/HD patients and the subpopulation of AD/HD patients suffering from comorbid tic disorders. Examples of compounds that fall into the anti-AD/HD (≠DA, NE) subclass include: (1) compounds with dopamine and GABA stimulating activity, (2) compounds with dopamine stimulating activity and glutaminergic inhibitory activity, (3) compounds with dopamine and GABA stimulating activity and glutaminergic inhibitory activity, (4) compounds with norepinephrine and GABA stimulating activity, (5) compounds with norepinephrine stimulating activity and glutaminergic inhibitory activity, (6) compounds with norepinephrine and GABA stimulating activity and glutaminergic inhibitory activity, and (7) compounds with norepinephrine stimulating activity and dopamine inhibitory activity. The anti-AD/HD ((≠DA, NE) compounds can exhibit additional pharmacological activities not listed herein. A specific example of a compound that falls into the anti-AD/HD (≠DA, NE) subclass is milnacipran and its analogs.

The term “anti-AD/HD properties” as used herein means therapeutic and/or prophylactic activity towards AD/HD. By therapeutic activity is meant eradication or amelioration of the underlying disorder being treated, e.g., eradication or amelioration of the underlying AD/HD and/or eradication or amelioration of one or more of the symptoms associated with the underlying disorder such that an improvement is observed in the patient's condition, notwithstanding that the patient may still be afflicted with the underlying disorder. For example, administration of a compound with anti-AD/HD properties to a patient suffering from AD/HD provides therapeutic benefit not only when the underlying AD/HD indication is eradicated or ameliorated, but also when the patient exhibits decreased inappropriate inattention and/or hyperactivity-impulsivity, even though the underlying AD/HD disorder may still be prevalent. By prophylactic activity is meant a delay or lack of development of the disorder in patients at a risk of developing AD/HD. Prophylactic benefits can be observed in patients who no longer exhibit symptoms of AD/HD but are administered the compounds of the present invention to prevent a relapse of AD/HD.

The term “anti-tic properties” is used herein to include therapeutic and/or prophylactic activity towards tic disorders in AD/HD patients. By therapeutic activity is meant eradication or amelioration of the underlying disorder being treated, e.g., eradication or amelioration of the underlying tic disorder, and/or eradication or amelioration of one or more of the symptoms associated with the underlying tic disorder such that an improvement is observed in the patient's condition, notwithstanding that the patient may still be afflicted with the underlying tic disorder. By prophylactic activity is meant a delay in the development of the disorder or the development of a less severe form of the disorder in patients at a risk of developing tic disorders.

This prophylactic activity of the present invention against tic disorders can be obtained in particular in AD/HD patients using dopamine stimulants like methylphenidate, pemoline, and dextroamphetamine. In a subclass of AD/HD patients, the use of dopamine stimulants results in the development of tic disorders or an aggravation of an existing tic disorder. Dopamine stimulants treat AD/HD by increasing dopamine activity in the central nervous system. However, increased dopamine activity is known to be one of the causes of tic disorders. Thus administration of dopamine stimulants to AD/HD patients occasionally causes the development of tic disorders or an aggravation of an existing tic disorder. In one embodiment of the invention, the compounds used to treat AD/HD patients decreases glutaminergic activity in addition to stimulating dopamine activity. While not intending to be bound by any particular theory of operation, it is believed that even though these compounds stimulate dopamine activity, the decrease in glutaminergic activity has an inhibitory effect on tics. In another embodiment of the invention, in addition to dopaminergic activity, the compounds useful in the present invention can have either GABA activity or noradrenergic activity. While not intending to be bound by any particular theory of operation, it is believed that both the GABA activity and noradrenergic activity have beneficial effects on tic disorders. Thus, the compounds of the present invention can have therapeutic and/or prophylactic effects on both the AD/HD and tics symptoms.

Due to the role of increased dopamine transmission in the pathophysiology of tic disorders, a particularly useful subclass of compounds is compounds that do not increase dopamine activity, but increase noradrenergic activity. As increased noradrenergic activity is not related to causation or aggravation of tic disorders, this subclass of compounds is particularly useful in treating AD/HD patients with comorbid tics disorders and AD/HD patients at a risk of developing comorbid tics disorders. In addition to the noradrenergic activity, this subclass of compounds would have at least one of the following activities: increased GABA activity, decreased glutaminergic activity, increased serotonin activity, or decreased dopamine activity. While not intending to be bound by any particular theory of operation, it is believed that the noradrenergic activity provides therapeutic and/or prophylactic benefits towards AD/HD and/or tic symptoms; the increase in GABA activity and decrease in glutaminergic or dopamine activity provides beneficial effects towards tic symptoms; and the increase in serotonin activity is beneficial towards the treatment of psychiatric disorders. Overall, it-is believed that by not increasing dopamine activity, this subclass of compounds is useful in treating AD/HD and the associated disorders without causing the development of tic disorder or without aggravating an existing tic disorder.

In one embodiment of the invention, the anti-AD/HD and anti-AD/HD (≠DA, NE) compounds used in the present invention are further characterized by an additional property, i.e., anti-psychiatric properties. This subclass of compounds can be used in treating AD/HD patients, in particular AD/HD patients suffering from comorbid psychiatric disorders. Also, this subclass of compounds can be used to treat AD/HD patients suffering from comorbid tic and psychiatric disorders.

The term “anti-psychiatric properties” is used herein to include therapeutic and/or prophylactic activities towards psychiatric disorders in AD/HD patients. By therapeutic activity is meant eradication or amelioration of the underlying disorder being treated, e.g., eradication or amelioration of the underlying psychiatric disorder, and/or eradication or amelioration of one or more of the symptoms associated with the underlying disorder such that an improvement is observed in the patient's condition, notwithstanding that the patient may still be afflicted with the underlying disorder. By prophylactic activity is meant a delay or lack of development of the disorder in patients at a risk of developing psychiatric disorders. For example, the compounds of the present invention can be used prophylactically in patients diagnosed with AD/HD, even though a diagnosis of psychiatric disorders has not been made. In these patients, the prophylactic activity conferred would be a delay in the development of psychiatric disorders or the development of a less severe form of psychiatric disorders.

### Use of SNRI-NMDA Compounds for Treatment of ADD, ADHD, Psychiatric Disorders, and Tic Disorders

One subclass of compounds useful in practicing the present invention is the serotonin norepinephrine reuptake inhibitor (SNRI) compounds with NMDA antagonism properties. Compounds in this subclass are referred herein to as “SNRI-NMDA compounds.” The SNRI-NMDA compounds used in the present invention can show an equal inhibition of norepinephrine and serotonin reuptake, or inhibit norepinephrine reuptake less than serotonin reuptake, or inhibit norepinephrine reuptake more than serotonin reuptake. The SNRI-NMDA compounds can be used to treat AD/HD patients, the subpopulation of AD/HD patients suffering from comorbid tic disorders, and the subpopulation of AD/HD patients suffering from comorbid tic and psychiatric disorders.

Particularly useful in the present invention are the SNRI-NMDA compounds that inhibit norepinephrine reuptake more than serotonin reuptake and are NMDA receptor antagonists. These compounds are referred to herein as “NSRI-NMDA compounds.” A particular example of a NSRI-NMDA compound useful in the present invention is milnacipran.

The NSRI-NMDA compounds generally have a NE:5-HT in the range of about 1.1-100:1. The term “NE:5-HT” herein refers to the ratio of inhibition of norepinephrine reuptake to serotonin reuptake. The NSRI-NMDA compounds are at least about 1.1 to about 100 times more effective at inhibiting norepinephrine reuptake than serotonin reuptake. NSRI-NMDA compounds having a NE:5-HT ratio in the range of about 2:1 to about 10:1 may be particularly effective.

Various techniques are known in the art to determine the NE:5-HT of a particular compound. In one embodiment, the ratio can be calculated from IC₅₀ data for NE and 5-HT reuptake inhibition. For example, it has been reported that for milnacipran the IC₅₀ of norepinephrine reuptake is 100 nM, whereas the IC₅₀ serotonin reuptake inhibition is 200 nM. See Moret et al., 1985, Neuropharmacology 24(12): 1211-1219; Palmier et al., 1989, Eur J Clin Pharmacol 37: 235-238. Therefore, the NE:5-HT reuptake inhibition ratio for milnacipran based on this data is 2:1. Of course, other IC values such as IC₂₅, IC₇₅, etc. could be used, so long as the same IC value is being compared for both norepinephrine and serotonin. The concentrations necessary to achieve the desired degree of inhibition (i.e., IC value) can be calculated using known techniques either in vivo or in vitro. See Sanchez et al., 1999, Cellular and Molecular Neurobiology 19(4): 467-489; Turcotte et al., 2001, Neuropsychopharmacology 24(5): 511-521; Moret et al., 1985, Neuropharmacology 24(12): 1211-1219; Moret et al., 1997, J Neurochem. 69(2): 815-822; Bel et al., 1999, Neuropsychopharmacology 21(6): 745-754; and

Palmier et al., 1989, Eur J Clin Pharmacol 37: 235-238.

The SNRI-NMDA compounds suitable for the present invention can have IC₅₀ values at the NMDA receptor from about 1 nM-100 μM. For example, milnacipran has been reported to have an IC₅₀ value of about 6.3 μM. The NMDA receptor antagonistic properties of milnacipran and its derivatives are described in Shuto et al., 1995, J Med. Chem., 38: 2964-2968; Shuto et al., 1996, J Med Chem. 39: 4844-4852; Shuto et al., 1998, J Med. Chem. 41: 3507-3514; and Shuto et al., 2001, Jpn. J Pharmacol. 85: 207-213. Methods for determining the antagonism and affinity for antagonism are disclosed in Shuto et al., 1995, J Med. Chem. 38: 2964-2968; Shuto et al., 1996, J Med. Chem. 39: 4844-4852; Shuto et al., 1998, J Med. Chem. 41: 3507-3514; Noguchi et al., 1999, Synapse 31: 87-96; and Shuto et al., 2001, Jpn. J Pharmacol. 85: 207-213.

Milnacipran derivatives disclosed in WO95/22521; U.S. Pat. No. 5,621,142; U.S. Pat. No. 4,478,836; Shuto et al., 1995, J Med. Chem. 38: 2964-2968; Shuto et al., 1996, Med. Chem. 39: 4844-4852; Shuto et al., 1998, J Med. Chem. 41: 3507-3514; Noguchi et al., 1999, Synapse 31: 87-96; and Shuto et al., 2001, Jpn. J Pharmacol. 85: 207-213 that inhibit both NE and 5-HT reuptake and have NMDA antagonistic properties can be used to practice the present invention. These references are hereby incorporated by reference in their entirety.

The chemical structure of milnacipran, cis-(±)-2-(aminomethyl)-N,N-diethyl-1-phenyl-yclopropanecarboxamide, is as follows: 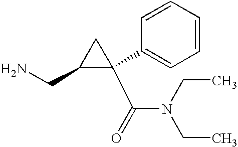
Milnacipran is also known in the art as F2207, TN-912, dalcipran, midalcipran, and midalipran. The ratio of NE:5-HT reuptake inhibition of milnacipran is 2:1. See Moret et al., 1985, Neuropharmacology 24(12): 1211-1219; Palmier et al., 1989, Eur J Clin Pharmacol 37: 235-238. Milnacipran and methods for its synthesis are described in U.S. Pat. No. 4,478.836, which is hereby incorporated by reference in its entirety. Additional information regarding milnacipran may be found in the Merck Index, 12^{th} Edition, at entry 6281. Quite significantly, milnacipran has been used as an antidepressant in approximately 400,000 patients, and is known to be nontoxic in humans. In clinical trials at dosages of 100 mg/day or 200 mg/day, milnacipran was well tolerated and usually produced no more adverse effects than placebo (Spencer and Wilde, 1998, Drugs 56(3): 405-427).

Those of skill in the art will recognize that SNRI-NMDA compounds such as milnacipran may exhibit the phenomena of tautomerism, conformational isomerism, geometric isomerism and/or optical isomerism. It should be understood that the invention encompasses any tautomeric, conformational isomeric, optical isomeric and/or geometric isomeric forms of the SNRI-NMDA compounds having one or more of the utilities described herein, as well as mixtures of these various different forms. For example, as is clear from the above structural diagram, milnacipran is optically active. It has been reported in the literature that the dextrogyral enantiomer of milnacipran is about twice as active in inhibiting norepinephrine and serotonin reuptake than the racemic mixture, and that the levrogyral enantiomer is much less potent (see, e.g., Spencer and Wilde, 1998, supra; Viazzo et al., 1996, Tetrahedron Lett. 37(26): 4519-4522; Deprez et al., 1998, Eur. J Drug Metab. Pharmacokinet. 23(2): 166-171). Accordingly, milnacipran may be administered in enantiomerically pure form (e.g., the pure dextrogyral enantiomer) or as a mixture of dextogyral and levrogyral enantiomers, such as a racemic mixture. Unless specifically noted otherwise, the term “milancipran” as used herein refers to both enantiomerically pure forms of milnacipran as well as to mixtures of milnacipran enantiomers. Methods for separating and isolating the dextro- and levrogyral enantiomers of milnacipran and other SNRI-NMDA compounds are well-known (see, e.g., Grard et al., 2000, Electrophoresis 2000 21: 3028-3034).

It will also be appreciated that in many instances the SNRI-NMDA compounds may metabolize to produce active SNRI-NMDA compounds. The use of active metabolites is also within the scope of the present invention.

The inventors have discovered that SNRI-NMDA compounds, particularly NSRI-NMDA compounds, are effective in treating the symptoms associated with AD/HD and also the comorbid psychiatric and tic disorders. While not intending to be bound by any particular theory of operation, it is believed that the SNRI-NMDA compounds inhibit reuptake of norepinephrine which causes an improvement in attention and/or impulsivity-hyperactivity in AD/HD patients. These compounds are believed to not exacerbate tic disorder due to the effect on norepinephrine and a lack of effect on dopamine. Also, the tic blocking ability of these compounds is believed to be via the antagonistic effects at the NMDA receptor. In addition, the inhibition of reuptake of serotonin is believed to produce beneficial effects on the comorbid psychiatric disorders. Overall, due to the inhibition of the reuptake of norepinephrine and serotonin and the antagonistic activity at the NMDA receptor, the SNRI-NMDA compounds are useful in improving attention and impulsivity-hyperactivity, treating psychiatric disorders, and blocking tic disorders in AD/HD patients.

### Use of Triple Reuptake Inhibitors for Treatment of AD/HD, Psychiatric Disorders, and Tic Disorders

Another subclass of anti-AD/HD compounds useful in the present invention is the SNRI compounds that inhibit the reuptake of dopamine, in addition to inhibiting the reuptake of serotonin and norepinephrine. This subclass of compounds is referred to herein as the triple reuptake inhibitors. The triple reuptake inhibitors are effective in the treatment of a subpopulation of AD/HD patients that also suffer from co-morbid tic disorders. In addition, this subclass of compounds can be used to treat the subpopulation of AD/HD patients suffering from comorbid tic and psychiatric disorders. Compounds from this subclass that are useful in the present invention include didesmethylsibutramine, sibutramine, NS-2359, NS-2389, BTS-74398, and BSF-74681.

Triple reuptake inhibitors particularly useful in the present invention can have a ratio of inhibition of norepinephrine reuptake to dopamine reuptake (“NE:DA”) in the range of about 1.1-100:1. That is, these compounds inhibit the reuptake of norepinephrine greater than the reuptake of dopamine.

The triple reuptake inhibitors have several advantages over the currently available dopamine stimulating drugs therapy for AD/HD. The compounds in this subclass have increased dopamine activity that can produce positive effects on the symptoms of AD/HD. However, as mentioned above, increased dopamine activity can contribute to the pathophysiology of tic disorders. This drawback of the dopamine stimulating drugs is avoided in the present invention as the suitable triple reuptake inhibitors inhibit reuptake of norepinephrine greater than dopamine. The norepinephrine activity is believed to have an inhibitory effect on tic disorders. In addition, the norepinephrine activity can produce beneficial effects on the symptoms of AD/HD.

As mentioned above, the triple reuptake inhibitors suitable for the present invention are characterized by a lack of ability to cause or exacerbate tic symptoms. The effect of these compounds on tic disorders can be evaluated in animal models of tics. Several animal models of tic disorders are well known in the art. One example of an animal of tic disorders is McGrath et al., 2000, Brain Research, 877: 23-30. In one embodiment of the invention, triple reuptake inhibitors suitable for the invention do not cause a statistically significant increase in tic-like symptoms in the animal model described by McGrath et al., 2000.

### Adjunctive Administration

The compounds of the present invention, such as, for example, milnacipran, can be administered adjunctively with other active compounds such as typical & atypical antipsychotics, dopamine depleters, GABA agonists, and histamine-3 antagonists. Specific examples of compounds that can be adjunctively administered with the compounds of the present invention include, but are not limited to fluphenazine, pimozide, haloperidol, risperidone, ziprasidone, ziprasidone, thiothixene, trifluoperazine, molindone, tetrabenazine, topiramate, clonazepam, and Perceptin™. By adjunctive administration is meant simultaneous administration of the compounds, in the same dosage form, simultaneous administration in separate dosage forms, and separate administration of the compounds. For example, milnacipran can be simultaneously administered with fluphenazine, wherein both milnacipran and fluphenazine are formulated together in the same tablet. Alternatively, milnacipran could be simultaneously administered with fluphenazine, wherein both the milnacipran and fluphenazine are present in two separate tablets. In another alternative, milnacipran could be administered first followed by the administration of fluphenazine, or vice versa.

### Formulation and Routes of Administration

The compounds useful in the present invention, or pharmaceutically acceptable salts thereof, can be delivered to a patient using a wide variety of routes or modes of administration. Suitable routes of administration include, but are not limited to, inhalation, transdermal, oral, rectal, transmucosal, intestinal and parenteral administration, including intramuscular subcutaneous, and intravenous injections.

The term “pharmaceutically acceptable salt” means those salts which retain the biological effectiveness and properties of the compounds used in the present invention, and which are not biologically or otherwise undesirable. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compound contains a carboxy group, it may be converted into a pharmaceutically acceptable salt with inorganic or organic bases. Examples of suitable bases include sodium hydroxide, potassium hydroxide, ammonia, cyclohexylamine, dicyclohexyl-amine, ethanolamine, diethanolamine and triethanolamine.

The compounds, or pharmaceutically acceptable salts thereof, may be administered singly, and/or in cocktails combined with other therapeutic agents. Of course, the choice of therapeutic agents that can be co-administered with the compounds of the invention will depend, in part, on the condition being treated.

The active compounds of the present invention (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the active compounds may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For administration orally, the compounds may be formulated as a sustained release preparation. Numerous techniques for formulating sustained release preparations are described in the following references—U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491. Specifically, sustained release formulations of milnacipran are described in WO 98/08495. These references are hereby incorporated herein by reference in their entireties.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the active compound(s) may be conveniently delivered in the form of an aerosol spray presentation from pressurized packs or a nebulizer, with the use of a suitable propellant, e.,g, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compound(s) may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation or transcutaneous delivery (for example subcutaneously or intramuscularly), intramuscular injection or a transdermal patch. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Effective Dosages

Pharmaceutical compositions suitable for use in the present invention include compositions wherein the active ingredient is contained in a therapeutically or prophylactically effective amount, i.e., in an amount effective to achieve therapeutic or prophylactic benefit, as previously discussed. Of course, the actual amount effective for a particular application will depend, inter alia, on the condition being treated and the route of administration. Determination of an effective amount is well within the capabilities of those skilled in the art, especially in light of the disclosure herein.

Therapeutically effective amounts for use in humans can be determined from animal models. For example, a dose for humans can be formulated to achieve circulating concentration that has been found to be effective in animals. Examples of animal models suitable for this purpose are described in Russell et al., 2000, Behavioral Brain Research, 117: 69-74; Russell, 2001, Metab. Brain Dis., 16: 143-149; Sagvolden et al., 1992, Behav. Neural Biol., 58: 103-112; and McGrath et al., 2000, Brain Research, 877: 23-30.

Effective amounts of SNRI-NMDA compounds and triple reuptake inhibitors for use in humans can also be determined from human data in which the SNRI-NMDA compounds and triple reuptake inhibitors were used to treat other diseases. The amount administered can be the same amount administered to treat the other disease or can be an amount lower than the amount administered to treat the other disease. For example, 50 mg -400 mg/day of milnacipran is administered to treat depression. Thus, either 50 mg -400 mg/day or a lower dose can be administered for practicing the present invention.

Patient doses for oral administration of the compounds of the present invention typically range from about 1 μg-1 gm/day. For example, for the treatment of AD/HD and associated psychiatric disorders and/or associated tic disorders with milnacipran the dosage range is typically from 25 mg -400 mg/day, more typically from 100 mg -250 mg/day. The dosage may be administered once per day or several or multiple times per day. The amount of the compound administered to practice methods of the present invention will of course, be dependent on the subject being treated, the severity of the affliction, the manner of administration and the judgment of the prescribing physician. The dose used to practice the invention can produce the desired therapeutic or prophylactic effects, without producing serious side effects.

Specific embodiments of the present invention include:

[1] One embodiment of the present invention includes a method of treating attention deficit/hyperactivity disorder (AD/HD) and/or tic disorders associated therewith in an animal subject. The method includes administering to an animal subject suffering from AD/HD and comorbid tic disorder, an effective amount of an anti-AD/HD compound or a pharmaceutically acceptable salt thereof.

[2] Another embodiment of the present invention provides the method according to embodiment [1], wherein said compound is further characterized by anti-psychiatric properties.

[3] Another embodiment of the present invention provides the method according to embodiment [1], wherein the pharmacological activities of said compound are selected from the group consisting of dopamine stimulation, α2 agonistic activity, inhibition of norepinephrine reuptake, dopamine antagonistic activity, increased GABA activity in the central nervous system, decreased glutaminergic activity, and increased serotonin activity.

[4] Another embodiment of the present invention provides the method according to embodiment [1], wherein AD/HD is treated.

[5] Another embodiment of the present invention provides the method according to embodiment [1], wherein the tic disorders associated with AD/HD are treated.

[6] Another embodiment of the present invention provides the method according to embodiment [1], wherein the compound is administered adjunctively with fluphenazine, pimozide, haloperidol, risperidone, ziprasidone, ziprasidone thiothixene, trifluoperazine, molindone, tetrabenazine, topiramate, clonazepam, or Perceptin™.

[7] Another embodiment of the present invention provides the method according to embodiment [1], wherein the animal subject is a human.

[8] Another embodiment of the present invention provides a method of treating AD/HD, tic disorders associated therewith, or a combination thereof, in an animal subject. The method includes administering to an animal subject suffering from AD/HD, an effective amount of an anti-AD/HD (≠DA, NE) compound or a pharmaceutically acceptable salt thereof.

[9] Another embodiment of the present invention provides the method according to embodiment [8], wherein said compound is further characterized by anti-psychiatric properties.

[10] Another embodiment of the present invention provides the method according to embodiment [8], wherein the pharmacological activities of said compound are selected from the group consisting of dopamine stimulation, α2 agonistic activity, inhibition of norepinephrine reuptake, dopamine antagonistic activity, increased GABA activity in the central nervous system, decreased glutaminergic activity, and increased serotonin activity.

[11] Another embodiment of the present invention provides the method according to embodiment [8], wherein AD/HD is treated.

[12] Another embodiment of the present invention provides the method according to embodiment [8], wherein the tic disorders associated with AD/HD are treated.

[13] Another embodiment of the present invention provides the method according to embodiment [8], wherein the compound is administered adjunctively with fluphenazine, pimozide, haloperidol, risperidone, ziprasidone, ziprasidone, thiothixene, trifluoperazine, molindone, tetrabenazine, topiramate, clonazepam, or Perceptin™.

[14] Another embodiment of the present invention provides the method according to embodiment [8], wherein the animal subject is a human.

[15] Another embodiment of the present invention provides a method of treating AD/HD, tic disorders associated therewith, or a combination thereof, in an animal subject. The method includes administering to an animal subject suffering from AD/HD and comorbid tic disorder, an effective amount of an anti-AD/HD (≠DA, NE) compound or a pharmaceutically acceptable salt thereof.

[16] Another embodiment of the present invention provides the method according to embodiment [15], wherein said compound is further characterized by anti-psychiatric properties.

[17] Another embodiment of the present invention provides the method according to embodiment [15], wherein the pharmacological activities of said compound are selected from the group consisting of dopamine stimulation, α2 agonistic activity, inhibition of norepinephrine reuptake, dopamine antagonistic activity, increased GABA activity in the central nervous system, decreased glutaminergic activity, and increased serotonin activity.

[18] Another embodiment of the present invention provides the method according to embodiment [15], wherein AD/HD is treated.

[19] Another embodiment of the present invention provides the method according to embodiment [15], wherein the tic disorders associated with AD/HD are treated.

[20] Another embodiment of the present invention provides the method according to embodiment [15], wherein the compound is administered adjunctively with fluphenazine, pimozide, haloperidol, risperidone, ziprasidone, ziprasidone, thiothixene, trifluoperazine, molindone, tetrabenazine, topiramate, clonazepam, or Perceptin™.

[21] Another embodiment of the present invention provides the method according to embodiment [15], wherein the animal subject is a human.

[22] Another embodiment of the present invention provides a method of treating AD/HD, tic disorders associated therewith, or a combination thereof, in an animal subject. The method includes administering to an animal subject suffering from AD/HD, an effective amount of milnacipran, or a pharmaceutically acceptable salt thereof.

[23] Another embodiment of the present invention provides a method of treating AD/HD, tic disorders associated therewith, or a combination thereof, in an animal subject. The method includes administering to an animal subject suffering from AD/HD and comorbid tic disorders, an effective amount of milnacipran, or a pharmaceutically acceptable salt thereof.

[24] Another embodiment of the present invention provides the method according to embodiment [22] or [23], wherein the milnacipran is formulated in a sustained release dosage form.

[25] Another embodiment of the present invention provides a kit that includes an anti-AD/HD compound or a pharmaceutically acceptable salt thereof, and instructions teaching a method of use according to embodiment [1].

[26] Another embodiment of the present invention provides a kit of embodiment [25] in which the compound or salt thereof is packaged in unit dosage form.

[27] Another embodiment of the present invention provides the kit of embodiment [25] in which the compound is milnacipran.

[28] Another embodiment of the present invention provides a kit that includes an anti-AD/HD (≠DA, NE) compound or a pharmaceutically acceptable salt thereof, and instructions teaching a method of use according to anyone of embodiments [8] or [15].

[29] Another embodiment of the present invention provides the kit of embodiment [28], in which the compound or salt thereof is packaged in unit 30.

[30] Another embodiment of the present invention provides the kit of embodiment [28], in which the compound is milnacipran.

Additional specific embodiments of the present invention include:

[31] One embodiment of the present invention provides a method of treating attention deficit/hyperactivity disorder (AD/HD), tic disorders associated with attention deficit/hyperactivity disorder (AD/HD), or a combination thereof, in a mammal. The method includes administering to the mammal an effective amount of a compound that is an N-methyl-D-aspartate (NMDA) receptor antagonist, wherein the compound is also a selective norepinephrine (NE)-serotonin (5-HT) reuptake inhibitor (NSRI), a selective norepinephrine reuptake inhibitor (NERI), or a combination thereof.

[32] Another embodiment of the present invention provides a method of embodiment [31] wherein the N-methyl-D-aspartate (NMDA) receptor antagonist has a dissociation constant with the NMDA receptor of 50 micromolar (μM) or less.

[33] Another embodiment of the present invention provides a method of embodiment [31] wherein the N-methyl-D-aspartate (NMDA) receptor antagonist has a dissociation constant with the NMDA receptor of 20 micromolar (μM) or less.

[34] Another embodiment of the present invention provides a method of any one of embodiments [31]-[33] wherein the N-methyl-D-aspartate (NMDA) receptor antagonist is a non-competitive NMDA receptor antagonist, a competitive NMDA receptor antagonist, a glycine-site antagonist, a glutamate-site antagonist, an NR1 subunit antagonist, an antagonist of an NR2 subunit, (e.g., an NR2A-, NR2B, NR2C, or NR2-D antagonist), or an NR3 subunit antagonist. The antagonists of particular subunits may be selective or non-selective.

[35] Another embodiment of the present invention provides a method of any one of embodiments [31]-[33] wherein the NMDA receptor antagonist is a PCP-site NMDA receptor antagonist.

[36] Another embodiment of the present invention provides a method of any one of embodiments [31]-[34] wherein the selective norepinephrine reuptake inhibitor (NERI) has an IC₅₀ for inhibition of noradrenaline reuptake into synaptosomes from cerebral cortex of 1 micromolar (μM) or less.

[37] Another embodiment of the present invention provides a method of any one of embodiments [31]-[35] wherein the selective norepinephrine reuptake inhibitor (NERI) has an IC₅₀ for inhibition of noradrenaline reuptake into synaptosomes from cerebral cortex of 100 nanomolar (nM) or less.

[38] Another embodiment of the present invention provides a method of any one of embodiments [31]-[37] wherein the selective NSRI has an NE:5-HT reuptake inhibition ratio of at least about 1.

[39] Another embodiment of the present invention provides a method of any one of embodiments [31]-[37] wherein the selective NSRI has an NE:5-HT reuptake inhibition ratio of up to about 20.

[40] Another embodiment of the present invention provides a method of any one of embodiments [31]-[37] wherein the selective NSRI has an NE:5-HT reuptake inhibition ratio of about 1:1 to about 20:1.

[41] Another embodiment of the present invention provides a method of any one of embodiments [31]-[37] wherein the selective NSRI has an NE:5-HT reuptake inhibition ratio of about 1:1 to about 5:1.

[42] Another embodiment of the present invention provides a method of any one of embodiments [31]-[37] wherein the selective NSRI has an NE:5-HT reuptake inhibition ratio of about 1:1 to about 3:1.

[43] Another embodiment of the present invention provides a method of any one of embodiments [31]-[42] wherein the selective norepinephrine (NE)-serotonin (5-HT) reuptake inhibitor (NSRI) has limited post-synaptic receptor effects, such that the ki at each of adrenergic and cholinergic sites is greater than about 500 nanomolar (nM).

[44] Another embodiment of the present invention provides a method of any one of embodiments [31]-[43] wherein the compound is a compound of formula (I): 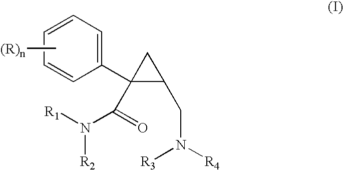
or sterioisomeric forms, mixtures of sterioisomeric forms, or pharmaceutically acceptable salts thereof wherein,
R is independently hydrogen, halo, alkyl, substituted alkyl, alkoxy, substituted alkoxy, hydroxy, nitro, amino, or substituted amino;
n is 1 or 2;
R₁ and R₂ are each independently hydrogen, alkyl, substituted alkyl, aryl, substituted aryl, cycloalkyl, substituted cycloalkyl, alkaryl, substituted alkaryl, heteroaryl, substituted heteroaryl, heterocycle, or substituted heterocycle; or
R₁ and R₂ can form a heterocycle, substituted heterocycle, heteroaryl, or substituted heteroaryl with the adjacent nitrogen atom;
R₃ and R₄ are each independently hydrogen, alkyl, or substituted alkyl; or
R₃ and R₄ can form a heterocycle, substituted heterocycle, heteroaryl, or substituted heteroaryl with the adjacent nitrogen atom.

[45] Another embodiment of the present invention provides a method of any one of embodiments [31]-[43] wherein the compound is a compound of formula (Ia): 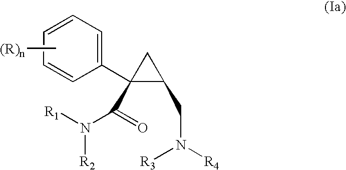
or sterioisomeric forms, mixtures of sterioisomeric forms, or pharmaceutically acceptable salts thereof wherein,
R is independently hydrogen, halo, alkyl, substituted alkyl, alkoxy, substituted alkoxy, hydroxy, nitro, amino, or substituted amino;
n is 1 or 2;
R₁ and R₂ are each independently hydrogen, alkyl, substituted alkyl, aryl, substituted aryl, cycloalkyl, substituted cycloalkyl, alkaryl, substituted alkaryl, heteroaryl, substituted heteroaryl, heterocycle, or substituted heterocycle; or
R₁ and R₂ can form a heterocycle, substituted heterocycle, heteroaryl, or substituted heteroaryl with the adjacent nitrogen atom;
R₃ and R₄ are each independently hydrogen, alkyl, or substituted alkyl; or
R₃ and R₄ can form a heterocycle, substituted heterocycle, heteroaryl, or substituted heteroaryl with the adjacent nitrogen atom.

[46] Another embodiment of the present invention provides a method of embodiment [45] wherein R is hydrogen.

[47] Another embodiment of the present invention provides a method of embodiment [45] wherein n is 1.

[48] Another embodiment of the present invention provides a method of embodiment [45] wherein R₁ is alkyl.

[49] Another embodiment of the present invention provides a method of embodiment [45] wherein R₁ is ethyl.

[50] Another embodiment of the present invention provides a method of embodiment [45] wherein R₂ is alkyl.

[51] Another embodiment of the present invention provides a method of embodiment [45] wherein R₂ is ethyl.

[52] Another embodiment of the present invention provides a method of embodiment [45] wherein R₃ is hydrogen.

[53] Another embodiment of the present invention provides a method of embodiment [45] wherein R₄ is hydrogen.

[54] Another embodiment of the present invention provides a method of embodiment [45] wherein the compound is (milnacipran) a compound of the formula: 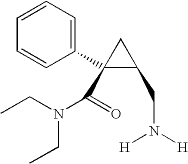
or sterioisomeric forms, mixtures of sterioisomeric forms, or pharmaceutically acceptable salts thereof.

[55] Another embodiment of the present invention provides a method of embodiment [54] wherein the compound of the formula recited therein (milnacipran) is administered up to about 400 mg/day.

[56] Another embodiment of the present invention provides a method of embodiment [54] wherein the compound of the formula recited therein (milnacipran) is administered in about 25 mg/day to about 250 mg/day.

[57] Another embodiment of the present invention provides a method of embodiment [54] wherein the compound of the formula recited therein (milnacipran) is administered one or more (e.g., 1, 2, 3, 4, or 5) times per day.

[58] Another embodiment of the present invention provides a method of any one of embodiments [31]-[57] wherein the N-methyl-D-aspartate (NMDA) receptor antagonist is not CGP 37-849, MK-801, or AP7; as disclosed in Behav. Neural. Biol. 60 p 224-(1993) and Exp. Brain Research 75 p 449 -(1989).

### EXAMPLES

### Example 1

### Assessment of the Efficacy of Milnacipran in an Animal Model of AD/HD

In this study, spontaneously hypertensive rats (SHR) are used as an animal model for AD/HD. The SHR animal model is described in Russell et al., 2000, Behavioral Brain Research, 117: 69-74; Russell, 2001, Metab. Brain Dis., 16: 143-149; and Sagvolden et al., 1992, Behav. Neural Biol., 58: 103-112. The study consists of two groups of rats: normal and SHR. Each group is further divided into two subgroups: placebo and milnacipran. The milnacipran subgroup is further divided into four subgroups and each subgroup is administered 5, 10, 25, or 50 mg/kg of milnacipran. The milnacipran is administered to the rats over a period of twenty-one days.

The rats are from the normal and SHR groups are trained in the delayed gratification response paradigm as described in Charrier et al., 1996, Pharmacology and Biochemistry and Behavior, 54: 149-157. In this paradigm, rats learn to choose between five food pellets delivered after 30 seconds and one food pellet delivered after 5 seconds. Normal rats learn to choose the five food pellets delivered after 30 seconds at a higher frequency. Compared to the normal rats it takes the rats in the SHR group a significantly longer time to learn to choose five food pellets delivered after 30 seconds at a higher frequency.

Following administration of milnacipran, the amount of time-required by the SHR rats to choose five food pellets delivered after 30 seconds at a higher frequency is reduced, approaching the amount of time required by the normal rats.

### Example 2

### Assessment of the Efficacy of Milnacipran in an Animal Model of Tic Disorder

The rats described in McGrath et al., 2000, Brain Research, 877: 23-30, are used to study the effects of milnacipran on tic disorders. The rats are divided into two groups: placebo and milnacipran. The milnacipran group is further divided into four subgroups and each subgroup is administered 5, 10, 25, or 50 mg/kg of milnacipran. The milnacipran is administered to the rats over a period of twenty-one days.

Abnormal behavior, specifically tic-like behavior are quantified before and after administration of milnacipran. Administration of milnacipran reduces the abnormal behavior such as climbing/leaping, gnawing, and other tic-like behaviors.

### Example 3

### Assessment of the Efficacy of Milnacipran in Patients with AD/HD and Comorbid Tic Disorder

This study is a randomized, double-blind, placebo-controlled trial of parallel groups. After the screening procedures and a 14-day washout period, the subjects are randomly assigned to receive either milnacipran or placebo for 8 weeks.

Before entry into the study, each patient undergoes a detailed clinical evaluation by a psychiatrist and/or a psychologist. The diagnosis of AD/HD and comorbid tic disorder is made on the basis of this interview.

Entry criteria includes age between 7 and 15 years, a DSM-IV diagnosis of AD/HD (any type), a DSM-IV tic disorder (any type), and a score of ≧1.5 standard deviation units for age and gender on the 10-item Conners hyperactivity index (Goyette et al., 1978, J. Abnorm. Child Psychol., 6: 221-236) rated by a teacher or parent.

Exclusion criteria includes evidence of major depression, generalized anxiety disorder, separation anxiety disorder, or psychotic symptoms. Children with moderate or more severe tic symptoms (Yale Global Tic Severity Scale [Leckman et al., 1989, J Am Acad Child Adolesc Psychiatry, 28: 566-573] total tic score of>22) or significant obsessive-compulsive symptoms (Children's Yale Brown Obsessive Compulsive Scale [Scahill et al., 1997, J Am Acad Child Adolesc Psychiatry, 36: 844-852] total score>15) are also excluded.

Before entry into the study the patients are tapered off their current medication. The participants in the study are randomly divided into two groups—milnacipran group and placebo control group. Each group consists of 5 patients. The patients in the milnacipran group are administered 1.5-2 mg/kg/day of milnacipran, for 8 weeks. The patients in the placebo group are administered a placebo for 8 weeks.

The patient's course is followed at visits with a primary clinician, who is blind to the patient's study group, every 2 weeks. The AD/HD rating scale, Clinical Global Impression global improvement score, and Yale Global Tic Severity Scale are used to follow the outcome measures.

The ADHD Rating Scale (DuPaul et al., 1998, Psychol Assess, 9: 436-444) is an 18-item measure of inattention and hyperactive/impulsive symptoms derived from DSM-IV. Each symptom was scored by the child's teacher from 0 to 3 (0=never [or rarely], 1=sometimes, 2=often, and 3=very often). The scale yields three scores: an inattention score and a hyperactive/impulsive score (range=0-27 for each score) and a total score (range=0-54).

In this study, the clinician who is blind to the subject's study group uses the Clinical Global Impression global improvement score to rate global improvement in AD/HD symptoms after an endpoint interview with the parent and the child and, if possible, a telephone conversation with the teacher during the week before the child's final study visit. The Clinical Global Impression global improvement score compares current symptom severity to baseline severity (Guy W (ed): ECDEU Assessment Manual for Psychopharmacol-ogy: Publication ADM 76-338. Washington, DC, US Department of Health, Education, and Welfare, 1976, pp 218-222; Conners et al., 1985, Psychopharmacol Bull; 21: 809-843). A score of 1 corresponds with very much improved and 2 with much improved, 3 denotes minimal change, and 4 represents no change. Scores of 5, 6, or 7 indicate deterioration (minimally worse, much worse, or very much worse, respectively). A score of much improved or very much improved, reflecting meaningful improvement in AD/HD symptoms both at school and at home, is counted as a positive response.

The Yale Global Tic Severity Scale is a semi structured clinical interview designed to measure current tic severity (Leckman et al., 1989, J Am Acad Child Adolesc Psychiatry, 28: 566-573). The scale yield three summary scores: total motor score (range=0-25), total phonic score (range=0-25), and total tic score (the sum of the motor and phonic scores).

After 8 weeks of treatment, the patients in the milnacipran group showed an improvement in the AD/HD Rating Scale, Clinical Global Improvement Scale, and Yale Global Tic Severity Scale.

Each of the patent applications, patents, publications, and other published documents mentioned or referred to in this specification is herein incorporated by reference in its entirety, to the same extent as if each individual patent application, patent, publication, and other published document was specifically and individually indicated to be incorporated by reference.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treating a disorder selected from the group consisting of attention deficit/hyperactivity disorder (AD/HD), tic disorders associated with attention deficit/hyperactivity disorder (AD/HD), and a combination thereof, in an animal subject comprising administering to the animal subject a composition comprising as the active ingredient an effective amount of a norepinephrine-serotonin reuptake inhibitor (NSRI) compound to treat the disorder, wherein the NSRI compound has a NE:5-HT reuptake inhibition ratio of between 1:1 and 10:1.

2. The method of claim 1, wherein the NSRI is also an N-methyl-D-aspartate (NMDA) receptor antagonist with a dissociation constant with the NMDA receptor of 50 micromolar (μM) or less.

3. The method of claim 2, wherein the N-methyl-D-aspartate (NMDA) receptor antagonist has a dissociation constant with the NMDA receptor of 20 micromolar (μM) or less.

4. The method of claim 1, wherein the N-methyl-D-aspartate (NMDA) receptor antagonist is a non-competitive NMDA receptor antagonist, a competitive NMDA receptor antagonist, a glycine-site antagonist, a glutamate-site antagonist, an NR1 subunit antagonist, an antagonist of an NR2 subunit, or an NR3 subunit antagonist.

5. The method of claim 1, wherein the NMDA receptor antagonist is a PCP-site NMDA receptor antagonist.

6. The method of claim 1, wherein the selective norepinephrine reuptake inhibitor (NERI) has an IC₅₀ for inhibition of noradrenaline reuptake into synaptosomes from cerebral cortex of 1 micromolar (μM) or less.

7. The method of claim 1, wherein the selective norepinephrine reuptake inhibitor (NERI) has an IC50 for inhibition of noradrenaline reuptake into synaptosomes from cerebral cortex of 1 nanomolar (nM) or less.

8. The method of claim 1, wherein the NSRI has an NE:5-HT reuptake inhibition ratio of about 1:1 to about 2:1.

9. The method of claim 1, wherein the NSRI has an NE:5-HT reuptake inhibition ratio of about 1:1 to about 5:1.

10. The method of claim 1, wherein the NSRI has an NE:5-HT reuptake inhibition ratio of about 1:1 to about 3:1.

11. The method of claim 1, wherein the NSRI has limited post-synaptic receptor effects, such that the ki at each of adrenergic and cholinergic sites is greater than about 500 nanomolar (nM).

12. The method of claim 1, wherein the compound is a compound of formula (I): 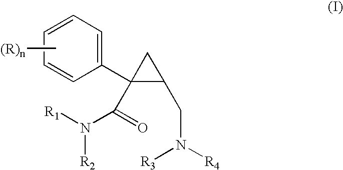
or sterioisomeric forms, mixtures of steriosomeric forms, or pharmaceutically acceptable salts thereof, wherein R is independently hydrogen, halo, alkyl, substituted alkyl, alkoxy, substituted alkoxy, hydroxy, nitro, amino, or substituted amino;
n is 1 or 2;
R₁ and R₂ are each independently hydrogen, alkyl, substituted alky, aryl, substituted aryl, cycloalkyl, substituted cycloalkyl, alkaryl, substituted alkaryl, heteroaryl, substituted heteroaryl, heterocycle, or substituted heterocycle; or
R₁ and R₂ can form a heterocycle, substituted heterocycle, heteroaryl, or substituted heteroaryl with the adjacent nitrogen atom;
R₃ and R₄ are each independently hydrogen, alkyl, or substituted alkyl; or
R₃ and R₄ can form a heterocycle, substituted heterocycle, heteroaryl, or substituted heteroaryl with the adjacent nitrogen atom.

13. The method of claim 1, wherein the compound is a compound of formula (Ia): 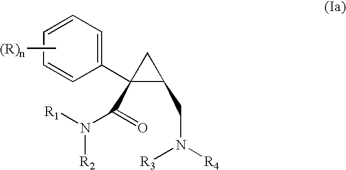
or sterioisomeric forms, mixtures of sterioisomeric forms, or pharmaceutically acceptable salts thereof wherein,
R is independently hydrogen, halo, alkyl, substituted alkyl, alkoxy, substituted alkoxy, hydroxy, nitro, amino, or substituted amino;
n is 1 or2;
R₁ and R₂ are each independently hydrogen, alkyl, substituted alkyl, aryl, substituted aryl, cycloalkyl, substituted cycloalkyl, alkaryl, substituted alkaryl, heteroaryl, substituted heteroaryl, heterocycle, or substituted heterocycle; or
R₁ and R₂ can form a heterocycle, substituted heterocycle, heteroaryl, or substituted heteroaryl with the adjacent nitrogen atom;
R₃ and R₄ are each independently hydrogen, alkyl, or substituted alkyl; or
R₃ and R₄ can form a heterocycle, substituted heterocycle, heteroaryl, or substituted heteroaryl with the adjacent nitrogen atom.

14. The method of claim 13, wherein the compound is milnacipran or sterioisomeric forms, mixtures of sterioisomeric forms, or pharmaceutically acceptable salts thereof.

15. The method of claim 14, wherein milnacipran is administered up to about 400 mg/day.

16. The method of claim 14, wherein milnacipran is administered in about 25 mg/day to about 250 mg/day.

17. The method of claim 1, wherein the N-methyl-D-aspartate (NMDA) receptor antagonist is not CGP 37-849, MK-801, or AP7.
